(19) United States
(12) Patent Application Publication
(10) Pub. No.: US 2022/0310233 A1
(43) Pub. Date: Sep. 29, 2022

(54) SYSTEM AND METHOD FOR GENERATING A MITOCHONDRIAL DYSFUNCTION NOURISHMENT PROGRAM

(71) Applicant: KPN INNOVATIONS, LLC., LAKEWOOD, CO (US)

(72) Inventor: Kenneth Neumann, LAKEWOOD, CO (US)

(73) Assignee: KPN INNOVATIONS, LLC., LAKEWOOD, CO (US)

(21) Appl. No.: 17/216,228

(22) Filed: Mar. 29, 2021

(51) Int. Cl. G16H 20/60; G16H 50/30; G16H 10/60; G16H 50/70; G16H 50/20; G16H 70/20; G16H 70/60; G16B 5/20; G16B 40/00

(52) U.S. Cl. CPC G16H 20/60; G16H 50/30; G16H 10/60; G16H 50/70; G16H 50/20; G16H 70/20; G16H 70/60; G16B 5/20; G16B 40/00

(57) ABSTRACT

A system for generating a mitochondrial dysfunction nourishment program includes a computing device configured to obtain a biological indicator, produce a mitochondrial profile as a function of the biological indicator, wherein producing further comprises identifying a probabilistic vector as a function of a medical examination, and producing the mitochondrial profile as a function of the probabilistic vector and the biological indicator using a profile machine-learning model, identify a biological modification as a function of the mitochondrial profile, wherein identifying the biological modification further comprises receiving a medical guideline, and identifying the biological modification as a function of the medical guideline and mitochondrial profile using a biological machine-learning model, determine an edible as a function of the biological modification, and generate a nourishment program as a function of the edible.

### FIELD OF THE INVENTION

The present invention generally relates to the field of artificial intelligence. In particular, the present invention is directed to a system and method for generating a mitochondrial dysfunction nourishment program.

### BACKGROUND

Current edible suggestion systems do not account for the status of an individual's mitochondrial functions. This leads to inefficiency of an edible suggestion system and a poor nutrition plan for the individual. This is further complicated by a lack of uniformity of nutritional plans, which results in dissatisfaction of individuals.

### SUMMARY OF THE DISCLOSURE

In an aspect, a system for generating a mitochondrial dysfunction nourishment program includes a computing device, the computing device configured to obtain a biological indicator, produce a mitochondrial profile as a function of the biological indicator, wherein producing further comprises identifying a probabilistic vector as a function of a medical examination, and producing the mitochondrial profile as a function of the probabilistic vector and the biological indicator using a profile machine-learning model, identify a biological modification as a function of the mitochondrial profile, wherein identifying the biological modification further comprises receiving a medical guideline, and identifying the biological modification as a function of the medical guideline and mitochondrial profile using a biological machine-learning model, determine an edible as a function of the biological modification, and generate a nourishment program as a function of the edible.

In another aspect, a method for generating a mitochondrial dysfunction nourishment program includes obtaining, by a computing device, a biological indicator, producing, by the computing device, a mitochondrial profile as a function of the biological indicator, wherein producing further comprises identifying a probabilistic vector as a function of a medical examination, and producing the mitochondrial profile as a function of the probabilistic vector and the biological indicator using a profile machine-learning model, identifying, by the computing device, a biological modification as a function of the mitochondrial profile, wherein identifying the biological modification further comprises receiving a medical guideline, and identifying the biological modification as a function of the medical guideline and mitochondrial profile using a biological machine-learning model, determining, by the computing device, an edible as a function of the biological modification, and generating, by the computing device, a nourishment program as a function of the edible.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating an exemplary embodiment of a system for generating a mitochondrial dysfunction nourishment program;

FIG. 2 is a block diagram of an exemplary embodiment of an epigenetic element according to an embodiment of the invention;

FIG. 3 is a block diagram of an exemplary embodiment of an edible directory according to an embodiment of the invention;

FIG. 4 is a block diagram of an exemplary embodiment of a medical examination according to an embodiment of the invention;

FIG. 5 is a block diagram of an exemplary embodiment of a machine-learning module;

FIG. 6 is a process flow diagram illustrating an exemplary embodiment of a method of generating a mitochondrial dysfunction nourishment program; and

FIG. 7 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for generating a mitochondrial dysfunction nourishment program. In an embodiment, this disclosure can obtain a biological indicator that relates to an individual's mitochondrial functioning. Aspects of the present disclosure can be used to produce a mitochondrial profile. This is so, at least in part, because this disclosure incorporates a probabilistic vector and a machine-learning model. Aspects of the present disclosure can also be used to identify a biological modification as a function of the mitochondrial profile. Aspects of the present disclosure can also be used to determine an edible as a function of the biological modification. Aspects of the present disclosure allow for generating a nourishment program. Exemplary embodiments illustrating aspects of the present disclosure are described below in the context of several specific examples.

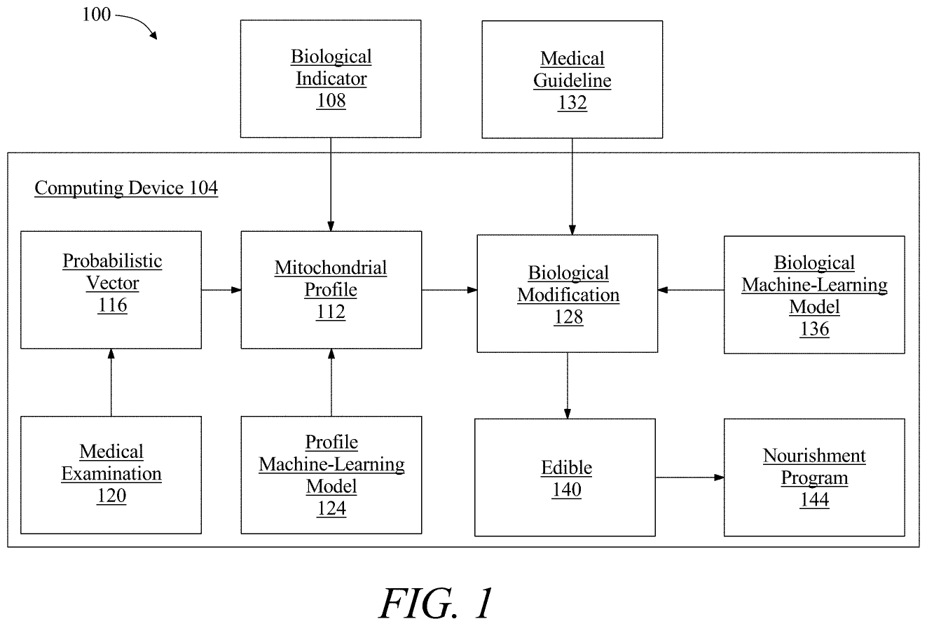

Referring now to FIG. 1, an exemplary embodiment of a system 100 for generating a mitochondrial dysfunction nourishment program is illustrated. System includes a computing device 104. Computing device 104 may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. computing device 104 may include a single computing device operating independently, or may include two or more computing device operating in concert, in parallel, sequentially or the like; two or more computing devices may be included together in a single computing device or in two or more computing devices. computing device 104 may interface or communicate with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. computing device 104 may include but is not limited to, for example, a computing device or cluster of computing devices in a first location and a second computing device or cluster of computing devices in a second location. computing device 104 may include one or more computing devices dedicated to data storage, security, distribution of traffic for load balancing, and the like. computing device 104 may distribute one or more computing tasks as described below across a plurality of computing devices of computing device, which may operate in parallel, in series, redundantly, or in any other manner used for distribution of tasks or memory between computing devices. computing device 104 may be implemented using a “shared nothing” architecture in which data is cached at the worker, in an embodiment, this may enable scalability of system 100 and/or computing device.

With continued reference to FIG. 1, computing device 104 may be designed and/or configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 1, computing device 104 is configured to obtain a biological indicator 108. As used in this disclosure a “biological indicator” is an element of data associated with an individual's biological system that denotes a health status, wherein a health status is a measure of the relative level of physical, mental, and/or social well-being of an individual. In an embodiment biological indicator may include an element that denotes an individual's genetic composition. As used in this disclosure a “genetic composition” is the composition of DNA unique to each individual. For example a first individual may have a first genetic composition, wherein a second individual may have a second genetic composition. In an embodiment biological indicator 108 may include an inheritance element. As used in this disclosure an “inheritance element” is an element associated with the inherited DNA from one or more parents of an individual. For example, and without limitation inheritance element may indicate that a particular region of an individual's DNA was inherited from a mother. As a further non-limiting example, inheritance element may indicate that a particular region of an individual's DNA was inherited from a father. Inheritance element may include one or more lineages, such as a DNA segment from a parent, grandparent, great grandparent, and the like thereof. Inheritance element may be associated with a diploid and/or haploid inheritance of a region of DNA. For example, a first region may be only inherited from a first parent, wherein a second region may be a combination of DNA inherited from the first parent and a second parent.

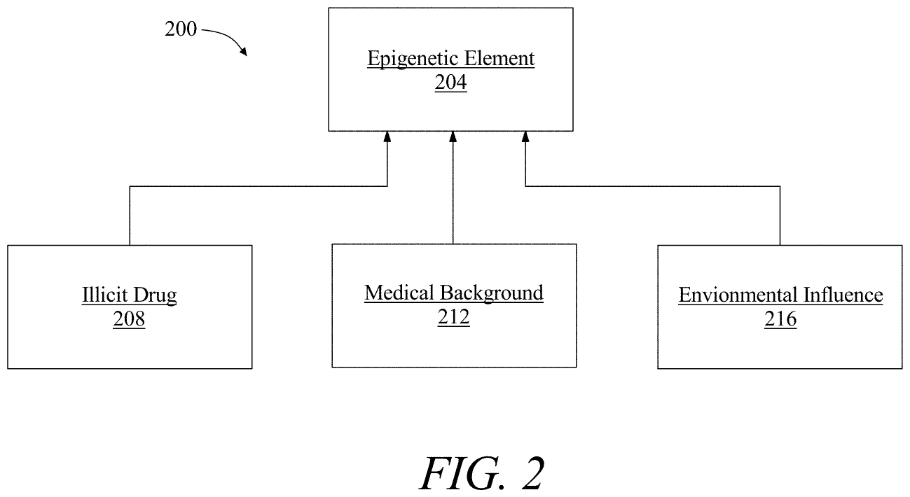

Still referring to FIG. 1, computing device 104 may obtain biological indicator 108 as a function of identifying a mutation component. As used in this disclosure a “mutation component” is a component associated with a mutation of an individual's DNA. As a non-limiting example, mutation component may denote that an individual's DNA should have a base pair combination of guanine and cytosine, wherein the base pair combination of the individual is guanine and thymine. Mutation component may include an epigenetic element, wherein an epigenetic element is an element relating to the change in a gene activity and/or expression as a function of one or more external factors, as described below in detail, in reference to FIG. 2. Computing device 104 may identify mutation component by receiving a spontaneity element. As used in this disclosure a “spontaneity element” is an element of date relating to the amount of spontaneity that a mutation may exhibit. For example, and without limitation a spontaneity element may denote that the expansion of the CGG triplet in the FMR-1 gene occurs in 1 of 1500 males and 1 of 2500 females. Computing device 104 may identify mutation component by determining a mutation rate. As used in this disclosure a “mutation rate” is a rate of mutation that commonly occurs at a DNA site. For example, and without limitation, mutation rate may include a 1.05×10⁻⁷/site/generation mutation rate for a guanine-cysteine base pair site to be mutated to an adenine-thymine base pair site. As a further non-limiting example, mutation rate may increase as a function of a low number of guanine and cytosine base pair sites. Computing device 104 may determine mutation rate as a function of spontaneity element and a mutation grouping. As used in this disclosure a “mutation grouping” is a category of mutations that affects DNA in a similar manner. For example, and without limitation, mutation grouping may include a point mutation. As used in this disclosure a “point mutation” is a mutation that affects one and/or very few nucleotides in a gene sequence. For example, and without limitation, point mutation may include a substitution of one or more nucleotides, an insertion of one or more nucleotides, and/or a deletion of one or more nucleotides. Mutation grouping may include a chromosomal mutation. As used in this disclosure a “chromosomal mutation” is a mutation of the inherited nucleic acid sequence of the genotype of an individual. For example, and without limitation, chromosomal mutation may include an inversion of a chromosome, a deletion of a chromosome, a duplication of a chromosome, and/or a translocation of a chromosome. Mutation grouping may include a copy number variation. As used in this disclosure a “copy number variation” is a mutation when the number of copies of a particular gene varies from one replication to another. For example, and without limitation, copy number variation may include gene amplification during a replication, expanding trinucleotide repeats as a function of a replication, and the like thereof

Still referring to FIG. 1, biological indicator 108 may include a biological sample. As used in this disclosure a “biological sample” is one or more biological specimens collected from an individual. Biological sample may include, without limitation, exhalate, blood, sputum, urine, saliva, feces, semen, and other bodily fluids, as well as tissue. Biological indicator 108 may include a biological sampling device. Biological indicator 108 may include one or more biomarkers. As used in this disclosure a “biomarker” is a molecule and/or chemical that identifies the status of an individual's health system. As a non-limiting example, biomarkers may include, lactate, pyruvate, creatine-kinase, retinol-binding protein, albumin, spermidine, putrescine, isovaleryl-carnitine, propionyl-carnitine, phosphatidyl-choline, FGF-21, GDF-15, NF-L, TFAM, RNA polymerase, DNA polymerase, POLG, TFB1M, TFB2M, NRF-1, NRF-2, PGC1-alpha, and the like thereof. As a further non-limiting example, biological indicator 108 may include datum from one or more devices that collect, store, and/or calculate one or more lights, voltages, currents, sounds, chemicals, pressures, and the like thereof that are associated with the individual's mitochondrial function. For example, and without limitation a device may include a/an magnetic resonance imaging device, magnetic resonance spectroscopy device, electroretinogram device, electrocardiogram device, ABER sensor, mass spectrometer, and the like thereof. Computing device 104 may obtain biological indicator 108 by receiving an input from a user. As used in this disclosure “input” is an element of datum that is obtained by the user. In an embodiment, and without limitation, input may include one or more inputs from a family member. For example, and without limitation, a brother, sister, mother, father, cousin, aunt, uncle, grandparent, child, friend, and the like thereof may enter to computing device 104 that an individual is often lethargic and/or low in energy.

Still referring to FIG. 1, computing device 104 is configured to produce a mitochondrial profile 112 as a function of biological indicator 108. As used in this disclosure a “mitochondrial profile” is a profile and/or estimation of an individual's mitochondrial health status. For example, and without limitation, mitochondrial profile 112 may denote that an individual's mitochondrial function is outputting lower than normal ATP quantities. As a further non-limiting example, mitochondrial profile 112 may denote that an individual's mitochondrial function is unable to store calcium ions in the endoplasmic reticulum of the mitochondrial matrix. As a further non-limiting example, mitochondrial profile 112 may indicate one or more alterations and/or variances in the capability of a mitochondrion to signal through mitochondrial reactive oxygen species, regulate the membrane potential, regulate cellular metabolism, regulate heme synthesis reactions, synthesize steroids, regulate hormonal signaling, regulate immune signaling, and/or regulating cellular quality control by reporting neuronal status towards microglia through specialized somatic-junctions. Mitochondrial profile 112 is produced as a function of identifying a probabilistic vector 116. As used in this disclosure a “probabilistic vector” is a data structure that represents one or more a quantitative values and/or measures probability associated with a mitochondrial function modification. For example, and without limitation, probabilistic vector 116 may indicate that an individual's mitochondrial function has a high probability of declining rapidly. As a further non-limiting example, probabilistic vector 116 may indicate that an individual's mitochondrial function has a high likelihood of severely altering the health status of the individual. Probabilistic vector 116 may indicate a probability as a function of an inheritance element and/or a mutation component. For example, probabilistic vector 116 may indicate that an individual has a high probability of mitochondrial functioning modification as a function of one or more nuclear genes from a mother, father, and/or other family lineage. As a further non-limiting example, probabilistic vector may indicate that an individual has a high probability of mitochondrial functioning modification as a function of one or more mitochondrial genes from a mother. As a further non-limiting example, probabilistic vector 116 may indicate that an individual has a high probability of mitochondrial functioning modification as a function of one or more mutation components and/or likelihood of mutations.

In an embodiment, and still referring to FIG. 1, a vector may be represented as an n-tuple of values, where n is one or more values, as described in further detail below; a vector may alternatively or additionally be represented as an element of a vector space, defined as a set of mathematical objects that can be added together under an operation of addition following properties of associativity, commutativity, existence of an identity element, and existence of an inverse element for each vector, and can be multiplied by scalar values under an operation of scalar multiplication compatible with field multiplication, and that has an identity element is distributive with respect to vector addition, and is distributive with respect to field addition. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm: l=\sqrt{Σᵢ₌₀ⁿaᵢ²}, where aᵢ is attribute number i of the vector. Scaling and/or normalization may function to make vector comparison independent of absolute quantities of attributes, while preserving any dependency on similarity of attributes.

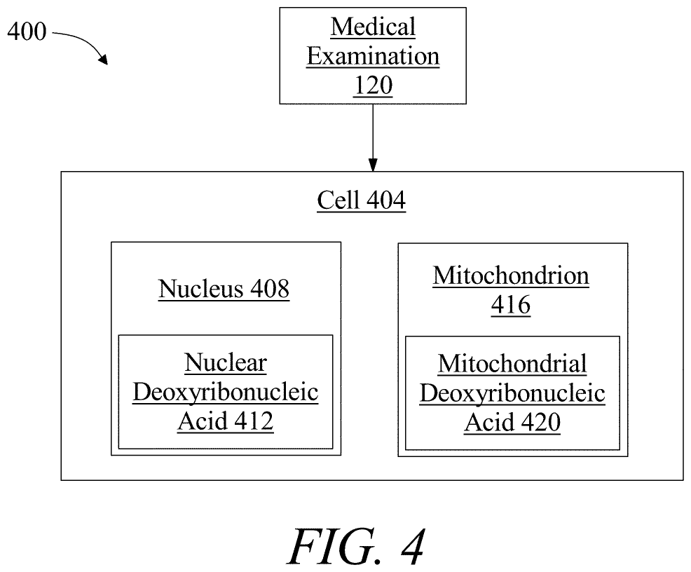

Still referring to FIG. 1, probabilistic vector 116 is identified as a function of a medical examination 120. As used in this disclosure a “medical examination” is an evaluation and/or estimation of the individual's health system. Medical examination 120 may include one or more examinations that identifies, characterizes, and/or otherwise evaluates the DNA responsible for mitochondrial functioning, as described below in detail, in reference to FIG. 4. In an embodiment, and without limitation, medical examination 120 may include a southern blot examination, a polymerase chain reaction examination, a genetic sequencing examination, a mass spectrometry examination, and the like thereof. Medical examination may be conducted by one or more informed advisors. As used in this disclosure “informed advisor” is an individual that is skilled in an area relating to the study of the health system of the individual. As a non-limiting example an informed advisor may include a medical professional who may assist and/or participate in the medical diagnosis, treatment, and/or guidance of an individual's health system including, but not limited to, family physicians, infectious disease physicians, mitochondrial specialists, geneticists, neurologists, physical therapists, primary care providers, and the like thereof. As a non-limiting example input may include an informed advisor that enters a medical assessment comprising a blood analysis, urine analysis, stool analysis, saliva analysis, skin analysis, and the like thereof. Additionally or alternatively, input may include one or more medical records and/or patient charts that identify an individual's previous medical history.

In an embodiment, and still referring to FIG. 1, computing device 104 may identify probabilistic vector 116 as a function of obtaining a mitochondrial deoxyribonucleic acid vector. As used in this disclosure a “mitochondrial deoxyribonucleic acid vector” is a measurable value associated with the mitochondrial deoxyribonucleic acid of an individual, wherein mitochondrial deoxyribonucleic acid is a nucleic acid, a polymeric biomolecule, and/or biopolymer found in the mitochondria of a cell, as described below in detail, in reference to FIG. 4. For example, and without limitation, a value of 22 may be associated with a point mutation of the mitochondrial deoxyribonucleic acid of an individual. Computing device 104 may receive a nuclear deoxyribonucleic acid vector. As used in this disclosure a “nuclear deoxyribonucleic acid vector” is a measurable value associated with the nuclear deoxyribonucleic acid of an individual, wherein nuclear deoxyribonucleic acid is a nucleic acid, a polymeric biomolecule, and/or biopolymer found in the nucleus of a cell, as described below in detail, in reference to FIG. 4. For example, and without limitation, a value of 91 may be associated with a chromosomal mutation of the nuclear deoxyribonucleic acid of an individual. Computing device 104 may identify probabilistic vector 116 as a function of the mitochondrial deoxyribonucleic acid vector and the nuclear deoxyribonucleic acid vector. For example, and without limitation, a mitochondrial deoxyribonucleic acid vector of 7 and a nuclear deoxyribonucleic acid vector of 5 may indicate that an individual has a low likelihood for developing a mitochondrial modification. As a further non-limiting example, a mitochondrial deoxyribonucleic acid vector of 81 and a nuclear deoxyribonucleic acid vector of 77 may indicate that an individual has a high likelihood for developing a mitochondrial modification.

Still referring to FIG. 1, computing device 104 produces mitochondrial profile 112 as a function of probabilistic vector 116 and biological indicator 108 using a profile machine-learning model 124. As used in this disclosure “profile machine-learning model” is a machine-learning model to produce a mitochondrial profile output given probabilistic vectors and biological indicators as inputs; this is in contrast to a non-machine learning software program where the commands to be executed are determined in advance by a user and written in a programming language. Profile machine-learning model 124 may include one or more profile machine-learning processes such as supervised, unsupervised, or reinforcement machine-learning processes that computing device 104 and/or a remote device may or may not use in the determination of mitochondrial profile 112. As used in this disclosure “remote device” is an external device to computing device 104. Profile machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train profile machine-learning process as a function of a profile training set. As used in this disclosure “profile training set” is a training set that correlates a probabilistic vector and/or biological indicator to a mitochondrial profile. For example, and without limitation, a probabilistic vector of a high likelihood for decreased ATP functioning and a biological indicator of a mutation of the C10ORF2 gene may relate to a mitochondrial profile of a decreased mitochondrial functioning. The profile training set may be received as a function of user-entered valuations of probabilistic vectors, biological indicators, and/or mitochondrial profiles. Computing device 104 may receive profile training set by receiving correlations of probabilistic vectors, and/or biological indicators that were previously received and/or determined during a previous iteration of determining mitochondrial profiles. The profile training set may be received by one or more remote devices that at least correlate a probabilistic vector and/or biological indicator to a mitochondrial profile. The profile training set may be received in the form of one or more user-entered correlations of a probabilistic vector and/or biological indicator to a mitochondrial profile. Additionally or alternatively, a user may include an informed advisor, wherein an informed advisor may include, without limitation, family physicians, infectious disease physicians, mitochondrial specialists, geneticists, neurologists, physical therapists, primary care providers, and the like thereof.

Still referring to FIG. 1, computing device 104 may receive profile machine-learning model 124 from a remote device that utilizes one or more profile machine learning processes, wherein a remote device is described above in detail. For example, and without limitation, a remote device may include a computing device, external device, processor, and the like thereof. Remote device may perform the profile machine-learning process using the profile training set to generate mitochondrial profile 112 and transmit the output to computing device 104. Remote device may transmit a signal, bit, datum, or parameter to computing device 104 that at least relates to mitochondrial profile 112. Additionally or alternatively, the remote device may provide an updated machine-learning model. For example, and without limitation, an updated machine-learning model may be comprised of a firmware update, a software update, a profile machine-learning process correction, and the like thereof. As a non-limiting example a software update may incorporate a new probabilistic vector that relates to a modified biological indicator. Additionally or alternatively, the updated machine learning model may be transmitted to the remote device, wherein the remote device may replace the profile machine-learning model with the updated machine-learning model and determine the mitochondrial profile as a function of the probabilistic vector using the updated machine-learning model. The updated machine-learning model may be transmitted by the remote device and received by computing device 104 as a software update, firmware update, or corrected profile machine-learning model. For example, and without limitation profile machine-learning model 124 may utilize a random forest machine-learning process, wherein the updated machine-learning model may incorporate a gradient boosting machine-learning process. Updated machine learning model may additionally or alternatively include any machine-learning model used as an updated machine learning model as described in U.S. Nonprovisional App. Ser. No. 17/106,658, filed on Nov. 30, 2020, and entitled “A SYSTEM AND METHOD FOR GENERATING A DYNAMIC WEIGHTED COMBINATION,” the entirety of which is incorporated herein by reference.

Still referring to FIG. 1, computing device 104 may produce mitochondrial profile 112 as a function of a classifier. A “classifier,” as used in this disclosure is a machine-learning model, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Computing device 104 and/or another device may generate a classifier using a classification algorithm, defined as a processes whereby a computing device 104 derives a classifier from training data. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers.

Still referring to FIG. 1, computing device 104 may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A)P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device 104 may then calculate a likelihood table by calculating probabilities of different data entries and classification labels. Computing device 104 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 1, computing device 104 may be configured to generate a classifier using a K-nearest neighbors (KNN) algorithm. A “K-nearest neighbors algorithm” as used in this disclosure, includes a classification method that utilizes feature similarity to analyze how closely out-of-sample- features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 1, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least one value. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm: l=\sqrt{Σᵢ₌₀ⁿaᵢ²}, where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

Still referring to FIG. 1, computing device 104 may identify mitochondrial profile 112 by identifying a mitochondrial dysfunction. As used in this disclosure an “mitochondrial dysfunction” is an ailment and/or collection of ailments associated with a mitochondrion such that individual's health system is impacted. As a non-limiting example, mitochondrial dysfunction may include autosomal dominant optic atrophy, Alpers disease, Barth syndrome, beta-oxidation defects, carnitine-acyl-carnitine deficiency, carnitine deficiency, complex I deficiency, complex II deficiency, complex III deficiency, complex IV deficiency, COX deficiency, complex V deficiency, CPT I deficiency, creatine deficiency syndromes, Co-enzyme Q10 deficiency, CPEO, CPT II deficiency, KSS, lactic acidosis, LBSL—leukodystrophy, LCA deficiency, LCHA deficiency, Leigh disease, Leber hereditary optic neuropathy, Luft disease, MAD, glutaric aciduria type II, MCAD, MERRF, MELAS, MEPAN, MIRAS, mitochondrial DNA depletion, mitochondrial encephalopathy, MNGIE, NARP, Pearson syndrome, POLG mutations, pyruvate carboxylase deficiency, PDC deficiency, SANDO, SCAD, SCHAD, TK2, myopathic form, VLCAD, and the like thereof. Mitochondrial dysfunction may be determined as a function of one or more dysfunction machine-learning models. As used in this disclosure, a “dysfunction machine-learning model” is a machine-learning model to produce a mitochondrial dysfunction output given biological indicator 108 and/or probabilistic vector 116 as inputs; this is in contrast to a non-machine learning software program where the commands to be executed are determined in advance by a user and written in a programming language. Dysfunction machine-learning model may include one or more dysfunction machine-learning processes such as supervised, unsupervised, or reinforcement machine-learning processes that computing device 104 and/or a remote device may or may not use in the determination of mitochondrial dysfunction. A dysfunction machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train dysfunction machine-learning process as a function of a dysfunction training set. As used in this disclosure, a “dysfunction training set” is a training set that correlates at least a mitochondrial enumeration and a mitochondrial effect to a mitochondrial dysfunction. As used in this disclosure, a “mitochondrial enumeration” is a measurable value associated with the biological indicator. As used in this disclosure, a “mitochondrial effect” is an impact and/or effect the biological indicator has on the mitochondrial functioning of an individual. As a non-limiting example a mitochondrial enumeration of 73 may be relate to a mitochondrial effect of decreased ATP production wherein a mitochondrial dysfunction of Leber hereditary optic neuropathy may be outputted. The dysfunction training set may be received as a function of user-entered valuations of mitochondrial enumerations, mitochondrial effects, and/or mitochondrial dysfunctions. Computing device 104 may receive dysfunction training set by receiving correlations of mitochondrial enumerations and/or mitochondrial effects that were previously received and/or determined during a previous iteration of determining mitochondrial dysfunctions. The dysfunction training set may be received by one or more remote devices that at least correlate a mitochondrial enumeration and/or mitochondrial effect to a mitochondrial dysfunction, wherein a remote device is an external device to computing device 104, as described above. The dysfunction training set may be received in the form of one or more user-entered correlations of a mitochondrial enumeration and mitochondrial effect to a mitochondrial dysfunction. Additionally or alternatively, a user may include an informed advisor, wherein an informed advisor may include, without limitation, family physicians, infectious disease physicians, mitochondrial specialists, geneticists, neurologists, physical therapists, primary care providers, and the like thereof.

Still referring to FIG. 1, computing device 104 may receive dysfunction machine-learning model from the remote device that utilizes one or more dysfunction machine learning processes, wherein a remote device is described above in detail. For example, and without limitation, a remote device may include a computing device, external device, processor, and the like thereof. The remote device may perform the dysfunction machine-learning process using the dysfunction training set to generate mitochondrial dysfunction and transmit the output to computing device 104. The remote device may transmit a signal, bit, datum, or parameter to computing device 104 that at least relates to mitochondrial dysfunctions. Additionally or alternatively, the remote device may provide an updated machine-learning model. For example, and without limitation, an updated machine-learning model may be comprised of a firmware update, a software update, a dysfunction machine-learning process correction, and the like thereof. As a non-limiting example a software update may incorporate a new mitochondrial enumeration that relates to a modified mitochondrial effect. Additionally or alternatively, the updated machine learning model may be transmitted to the remote device, wherein the remote device may replace the dysfunction machine-learning model with the updated machine-learning model and determine the mitochondrial dysfunction as a function of the mitochondrial enumeration using the updated machine-learning model. The updated machine-learning model may be transmitted by the remote device and received by computing device 104 as a software update, firmware update, or corrected dysfunction machine-learning model. For example, and without limitation dysfunction machine-learning model may utilize a Naïve bayes machine-learning process, wherein the updated machine-learning model may incorporate decision tree machine-learning process. Additionally or alternatively, dysfunction machine-learning model may determine the mitochondrial dysfunction as a function of one or more classifiers, wherein a classifier is described above in detail.

In an embodiment, and still referring to FIG. 1, computing device 104 may produce mitochondrial profile 112 by identifying a first probabilistic vector as a function of a first medical examination. For example, and without limitation, a first medical examination may identify a high likelihood for a mitochondrial modification as a function of a genetic sequencing examination. Computing device 104 may receive a second medical examination, for instance and without limitation as a function of a follow-up recommendation. As used in this disclosure a “follow-up recommendation” is a recommendation for an individual to receive a secondary and/or subsequent analysis to confirm, monitor, and/or evaluate the first medical examination results. For example and without limitation, follow-up recommendation may include a nuclear DNA test, mitochondrial DNA test, a mass spectrometric test, a blood test, a PCR analysis, a Southern blot test, and the like thereof. For example, and without limitation, follow-up recommendation may include recommending to a user a second PCR analysis 4 weeks after the first PCR analysis. Follow-up recommendation may include one or more tests and/or analyses wherein mitochondrial DNA may be analyzed to verify and/or confirm a first analysis. For example, and without limitation, follow-up recommendation may include a mitochondrial genetic analysis to confirm a first analysis of a mass spectrometric test. Computing device 104 may produce a second probabilistic vector as a function of the second medical examination. For example, and without limitation, a second probabilistic vector may indicate that the likelihood for a mitochondrial modification is less than the first probabilistic vector as a function of a mass spectrometric analysis. Computing device 104 may produce mitochondrial profile 112 as a function of first probabilistic vector and second probabilistic vector using profile machine-learning model 124, wherein profile machine-learning model 124 is described above.

Still referring to FIG. 1, computing device 104 is configured to identify a biological modification 128 as a function of mitochondrial profile 112. As used in this disclosure a “biological modification” is an effect that a mitochondrial profile has on the health system of an individual. For example, and without limitation, biological modification 128 may include one or more physical symptoms including, but not limited to, seizures, spasticity, blindness, liver dysfunction, cerebral degeneration, chronic fatigue, sarcopenia, cancer, poor growth, loss of muscle coordination, muscle weakness, autism, visual impairment, hearing impairment, heart disease, kidney disease, liver disease, respiratory disorders, thyroid dysfunction, and the like thereof. As a further non-limiting example, biological modification 128 may include one or more psychological symptoms including, but not limited to dementia, learning disabilities, social unawareness, and the like thereof. Computing device 104 identifies biological modification 128 by receiving a medical guideline 132. As used in this disclosure a “medical guideline” is a recommendation and/or guideline for mitochondrial functioning. As a non-limiting example, medical guideline 132 may include a recommendation that a mitochondrion should produce 100-150 mol/L of ATP per day. As a further non-limiting example medical guideline 132 may include a recommendation that a mitochondrion should store 100-200 nmol/L of calcium ions. Medical guideline 132 may include recommendations from one or more peer review sources such as scholarly peer reviews, government peer reviews, medical peer reviews, technical peer reviews, and the like thereof. Peer review sources may include, but are not limited to, The Journal of Biological Chemistry, Journal of Cell Biology, Molecular Cell, Journal of Cell Science, Cell Metabolism, PLOS Biology, The Journal of Physiology, and the like thereof. Medical guideline 132 may include recommendations from one or more informed advisor associations, such as a source of one or more committees, organizations, and/or groups capable of determining and/or organizing recommendations and/or guidelines. As a non-limiting example informed advisor associations may include the American Medical Association, Mitochondrial Disease Education & Research, United Mitochondrial Disease Foundation, Mitochondria Research Society, MitoAction, and the like thereof. Medical guideline 132 may include recommendations from one or more medical websites that establish guidelines for mitochondrial functions. As a non-limiting example medical websites may include, but are not limited to, UMDF, Medline Plus, Mayo Clinic, WebMD, Health.gov, and the like thereof.

Still referring to FIG. 1, biological modification 128 is identified as a function of medical guideline and mitochondrial profile 112 using a biological machine-learning model 136. As used in this disclosure a “biological machine-learning model” is a machine-learning model to produce a biological modification output given medical guidelines and/or mitochondrial profiles as inputs; this is in contrast to a non-machine learning software program where the commands to be executed are determined in advance by a user and written in a programming language. Biological machine-learning model may include one or more biological machine-learning processes such as supervised, unsupervised, or reinforcement machine-learning processes that computing device 104 and/or a remote device may or may not use in the determination of biological modification 128, wherein a remote device is an external device to computing device 104 as described above in detail. A biological machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train biological machine-learning process as a function of a biological training set. As used in this disclosure a “biological training set” is a training set that correlates at least medical guideline and mitochondrial profile to a biological modification. For example, and without limitation, medical guideline of reduced ROS species production and a mitochondrial profile associated with the mitochondrial dysfunction of CPEO may relate to a biological modification of visual myopathy, retinitis pigmentosa, dysfunction of the central nervous system and the like thereof. The biological training set may be received as a function of user-entered valuations of medical guidelines, mitochondrial profiles, and/or biological modifications. Computing device 104 may receive biological training set by receiving correlations of medical guidelines and/or mitochondrial profiles that were previously received and/or determined during a previous iteration of determining biological modifications. The biological training set may be received by one or more remote devices that at least correlate a medical guideline and mitochondrial profile to a biological modification, wherein a remote device is an external device to computing device 104, as described above. Biological training set may be received in the form of one or more user-entered correlations of a medical guideline and/or mitochondrial profile to a biological modification. Additionally or alternatively, a user may include an informed advisor, wherein an informed advisor may include, without limitation, family physicians, infectious disease physicians, mitochondrial specialists, geneticists, neurologists, physical therapists, primary care providers, and the like thereof

Still referring to FIG. 1, computing device 104 may receive biological machine-learning model 136 from a remote device that utilizes one or more biological machine learning processes, wherein remote device is described above in detail. For example, and without limitation, remote device may include a computing device, external device, processor, and the like thereof. Remote device may perform the biological machine-learning process using the biological training set to generate biological modification 128 and transmit the output to computing device 104. Remote device may transmit a signal, bit, datum, or parameter to computing device 104 that at least relates to biological modification 128. Additionally or alternatively, the remote device may provide an updated machine-learning model. For example, and without limitation, an updated machine-learning model may be comprised of a firmware update, a software update, a biological machine-learning process correction, and the like thereof. As a non-limiting example a software update may incorporate a new medical guideline that relates to a modified mitochondrial profile. Additionally or alternatively, the updated machine learning model may be transmitted to the remote device, wherein the remote device may replace the biological machine-learning model with the updated machine-learning model and determine the biological as a function of the mitochondrial profile using the updated machine-learning model. The updated machine-learning model may be transmitted by the remote device and received by computing device 104 as a software update, firmware update, or corrected biological machine-learning model. For example, and without limitation a biological machine-learning model 136 may utilize a neural net machine-learning process, wherein the updated machine-learning model may incorporate polynomial regression machine-learning process. Updated machine learning model may additionally or alternatively include any machine-learning model used as an updated machine learning model as described in U.S. Nonprovisional App. Ser. No. 17/106,658, filed on Nov. 30, 2020, and entitled “A SYSTEM AND METHOD FOR GENERATING A DYNAMIC WEIGHTED COMBINATION,” the entirety of which is incorporated herein by reference. In an embodiment, and without limitation, biological machine-learning model 136 may identify biological modification 128 as a function of one or more classifiers, wherein a classifier is described above in detail.

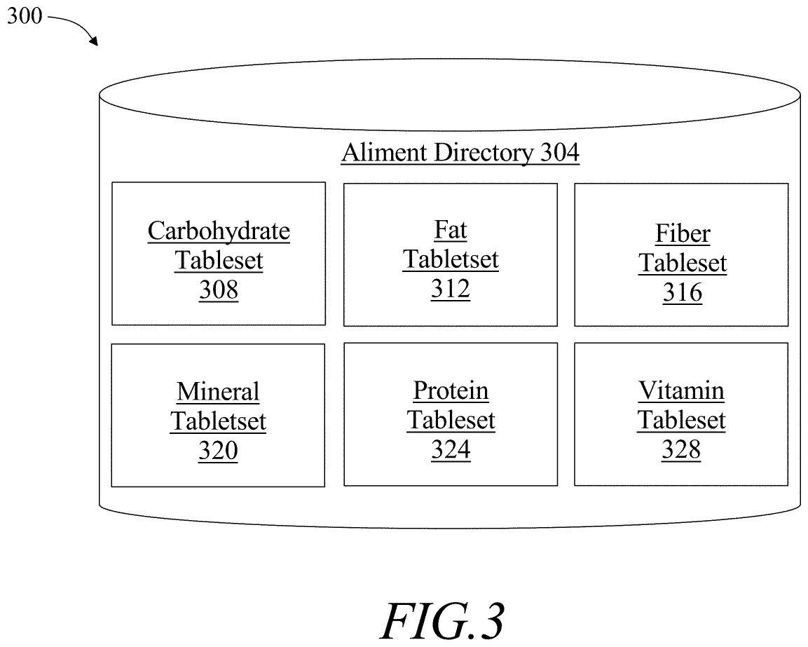

Still referring to FIG. 1, computing device determines an edible 140 as a function of biological modification 128. As used in this disclosure an “edible” is a source of nourishment that may be consumed by a user such that the user may absorb the nutrients from the source. For example and without limitation, an edible may include legumes, plants, fungi, nuts, seeds, breads, dairy, eggs, meat, cereals, rice, seafood, desserts, dried foods, dumplings, pies, noodles, salads, stews, soups, sauces, sandwiches, and the like thereof. Computing device 104 may determine edible 140 as a function of receiving a nourishment composition. As used in this disclosure a “nourishment composition” is a list and/or compilation of all of the nutrients contained in an edible. As a non-limiting example nourishment composition may include one or more quantities and/or amounts of total fat, including saturated fat and/or trans-fat, cholesterol, sodium, total carbohydrates, including dietary fiber and/or total sugars, protein, vitamin A, vitamin C, thiamin, riboflavin, niacin, pantothenic acid, vitamin b6, folate, biotin, vitamin B12, vitamin D, vitamin E, vitamin K, calcium, iron, phosphorous, iodine, magnesium, zinc, selenium, copper, manganese, chromium, molybdenum, chloride, and the like thereof. Nourishment composition may be obtained as a function of an edible directory, wherein an “edible directory” is a database of edibles that may be identified as a function of one or more metabolic components, as described in detail below, in reference to FIG. 3.

Still referring to FIG. 1, computing device 104 may produce a nourishment demand as a function of biological modification 128. As used in this disclosure a “nourishment demand” is requirement and/or necessary amount of nutrients required for a user to consume. As a non-limiting example, nourishment demand may include a user requirement of 600 mg of alpha-lipoic acid to be consumed per day. Nourishment demand may be determined as a function of receiving a nourishment goal. As used in this disclosure a “nourishment goal” is a recommended amount of nutrients that a user should consume. Nourishment goal may be identified by one or more organizations that relate to, represent, and/or study mitochondrial functions in humans, such as the American Medical Association, Mitochondrial Disease Education & Research, United Mitochondrial Disease Foundation, Mitochondria Research Society, MitoAction, and the like thereof.

Still referring to FIG. 1, computing device 104 identifies edible 140 as a function of nourishment composition, nourishment demand, and an edible machine-learning model. As used in this disclosure a “edible machine-learning model” is a machine-learning model to produce an edible output given nourishment compositions and nourishment demands as inputs; this is in contrast to a non-machine learning software program where the commands to be executed are determined in advance by a user and written in a programming language. Edible machine-learning model may include one or more edible machine-learning processes such as supervised, unsupervised, or reinforcement machine-learning processes that computing device 104 and/or a remote device may or may not use in the determination of edible 140, wherein a remote device is an external device to computing device 104 as described above in detail. An edible machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train edible machine-learning process as a function of an edible training set. As used in this disclosure an “edible training set” is a training set that correlates at least nourishment composition and nourishment demand to an edible. For example, and without limitation, nourishment composition of 900 mg of coenzyme Q₁₀ and a nourishment demand of 600 mg of coenzyme Q₁₀ as a function of a Barth syndrome may relate to an edible of beef heart. The edible training set may be received as a function of user-entered valuations of nourishment compositions, nourishment demands, and/or edibles. Computing device 104 may receive edible training set by receiving correlations of nourishment compositions and/or nourishment demands that were previously received and/or determined during a previous iteration of determining edibles. The edible training set may be received by one or more remote devices that at least correlate a nourishment composition and nourishment demand to an edible, wherein a remote device is an external device to computing device 104, as described above. Edible training set may be received in the form of one or more user-entered correlations of a nourishment composition and/or nourishment demand to an edible. Additionally or alternatively, a user may include an informed advisor, wherein an informed advisor may include, without limitation family physicians, infectious disease physicians, mitochondrial specialists, geneticists, neurologists, physical therapists, primary care providers, and the like thereof.

Still referring to FIG. 1, computing device 104 may receive edible machine-learning model from a remote device that utilizes one or more edible machine learning processes, wherein remote device is described above in detail. For example, and without limitation, remote device may include a computing device, external device, processor, and the like thereof. Remote device may perform the edible machine-learning process using the edible training set to generate edible 140 and transmit the output to computing device 104. Remote device may transmit a signal, bit, datum, or parameter to computing device 104 that at least relates to edible 140. Additionally or alternatively, the remote device may provide an updated machine-learning model. For example, and without limitation, an updated machine-learning model may be comprised of a firmware update, a software update, an edible machine-learning process correction, and the like thereof. As a non-limiting example a software update may incorporate a new nourishment composition that relates to a modified nourishment demand. Additionally or alternatively, the updated machine learning model may be transmitted to the remote device, wherein the remote device may replace the edible machine-learning model with the updated machine-learning model and determine the edible as a function of the nourishment demand using the updated machine-learning model. The updated machine-learning model may be transmitted by the remote device and received by computing device 104 as a software update, firmware update, or corrected edible machine-learning model. For example, and without limitation an edible machine-learning model may utilize a neural net machine-learning process, wherein the updated machine-learning model may incorporate polynomial regression machine-learning process. Updated machine learning model may additionally or alternatively include any machine-learning model used as an updated machine learning model as described in U.S. Nonprovisional App. Ser. No. 17/106,658, filed on Nov. 30, 2020, and entitled “A SYSTEM AND METHOD FOR GENERATING A DYNAMIC WEIGHTED COMBINATION,” the entirety of which is incorporated herein by reference. In an embodiment, and without limitation, edible machine-learning model may identify edible 140 as a function of one or more classifiers, wherein a classifier is described above in detail.

Still referring to FIG. 1, computing device 104 may identify edible as a function of a likelihood parameter. As used in this disclosure a “likelihood parameter” is a parameter that identities the probability of a user to consume an edible. As a non-limiting example likelihood parameter may identify a high probability that a user will consume an edible of fish. As a further non-limiting example likelihood parameter may identify a low probability that a user will consume an edible of chocolate. Likelihood parameter may be determined as a function of a user taste profile. As used in this disclosure a “user taste profile” is a profile of a user that identifies one or more desires, preferences, wishes, and/or wants that a user has. As a non-limiting example a user taste profile may include a user's preference for beef flavor and/or soft textured edibles. Likelihood parameter may be determined as a function of an edible profile. As used in this disclosure an “edible profile” is taste of an edible is the sensation of flavor perceived in the mouth and throat on contact with the edible. Edible profile may include one or more flavor variables. As used in this disclosure a “flavor variable” is a variable associated with the distinctive taste of an edible, wherein a distinctive may include, without limitation sweet, bitter, sour, salty, umami, cool, and/or hot. Edible profile may be determined as a function of receiving flavor variable from a flavor directory. As used in this disclosure a “flavor directory” is a database or other data structure including flavors for an edible. As a non-limiting example flavor directory may include a list and/or collection of edibles that all contain umami flavor variables. As a further non-limiting example flavor directory may include a list and/or collection of edibles that all contain sour flavor variables. Flavor directory may be implemented similarly to an edible directory as described below in detail, in reference to FIG. 3. Likelihood parameter may alternatively or additionally include any user taste profile and/or edible profile used as a likelihood parameter as described in U.S. Nonprovisional App. Ser. No. 17/032,080, filed on Sep. 25, 2020, and entitled “METHODS, SYSTEMS, AND DEVICES FOR GENERATING A REFRESHMENT INSTRUCTION SET BASED ON INDIVIDUAL PREFERENCES,” the entirety of which is incorporated herein by reference.

Still referring to FIG. 1, computing device 104 generates a nourishment program 144 as a function of edible 140. As used in this disclosure a “nourishment program” is a program consisting of one or more edibles that are to be consumed over a given time period, wherein a time period is a temporal measurement such as seconds, minutes, hours, days, weeks, months, years, and the like thereof. As a non-limiting example nourishment program 144 may consist of recommending avocados for 8 days. As a further non-limiting example nourishment program 144 may recommend fish for a first day, legumes for a second day, and pine nuts for a third day. Nourishment program 144 may include one or more diet programs such as paleo, keto, vegan, vegetarian, and the like thereof. Computing device 104 may develop nourishment program 144 as a function of a mitochondrial outcome. As used in this disclosure an “mitochondrial outcome” is an outcome that an edible may generate according to a predicted and/or purposeful plan. As a non-limiting example, mitochondrial outcome may include a treatment outcome. As used in this disclosure a “treatment outcome” is an intended outcome that is designed to at least reverse and/or eliminate biological modification 128, mitochondrial profile 112, biological indicator 108 and/or mitochondrial dysfunction. As a non-limiting example, a treatment outcome may include reversing the effects of the mitochondrial dysfunction autosomal dominant optic atrophy. As a further non-limiting example, a treatment outcome includes reversing the mitochondrial dysfunction of complex III deficiency. Mitochondrial outcome may include a prevention outcome. As used in this disclosure a “prevention outcome” is an intended outcome that is designed to at least prevent and/or avert biological modification 128, mitochondrial profile 112, biological indicator 108 and/or mitochondrial dysfunction. As a non-limiting example, a prevention outcome may include preventing the development of the mitochondrial dysfunction Pearson Syndrome.

Still referring to FIG. 1, computing device 104 may develop nourishment program 144 as a function of edible 140 and mitochondrial outcome using a nourishment machine-learning model. As used in this disclosure a “nourishment machine-learning model” is a machine-learning model to produce a nourishment program output given edibles and/or mitochondrial outcomes as inputs; this is in contrast to a non-machine learning software program where the commands to be executed are determined in advance by a user and written in a programming language. Nourishment machine-learning model may include one or more nourishment machine-learning processes such as supervised, unsupervised, or reinforcement machine-learning processes that computing device 104 and/or a remote device may or may not use in the development of nourishment program 144. Nourishment machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train nourishment machine-learning process as a function of a nourishment training set. As used in this disclosure a “nourishment training set” is a training set that correlates a mitochondrial outcome to an edible. The nourishment training set may be received as a function of user-entered edibles, mitochondrial outcomes, and/or nourishment programs. For example, and without limitation, a mitochondrial outcome of treating SCHAD may correlate to an edible of steak. Computing device 104 may receive nourishment training by receiving correlations of mitochondrial outcomes and/or edibles that were previously received and/or determined during a previous iteration of developing nourishment programs. The nourishment training set may be received by one or more remote devices that at least correlate a mitochondrial outcome and/or edible to a nourishment program, wherein a remote device is an external device to computing device 104, as described above. Nourishment training set may be received in the form of one or more user-entered correlations of a mitochondrial outcome and/or edible to a nourishment program. Additionally or alternatively, a user may include an informed advisor, wherein an informed advisor may include, without limitation family physicians, infectious disease physicians, mitochondrial specialists, geneticists, neurologists, physical therapists, primary care providers, and the like thereof.

Still referring to FIG. 1, computing device 104 may receive nourishment machine-learning model from the remote device that utilizes one or more nourishment machine learning processes, wherein a remote device is described above in detail. For example, and without limitation, a remote device may include a computing device, external device, processor, and the like thereof. The remote device may perform the nourishment machine-learning process using the nourishment training set to develop nourishment program 144 and transmit the output to computing device 104. The remote device may transmit a signal, bit, datum, or parameter to computing device 104 that at least relates to nourishment program 144. Additionally or alternatively, the remote device may provide an updated machine-learning model. For example, and without limitation, an updated machine-learning model may be comprised of a firmware update, a software update, a nourishment machine-learning process correction, and the like thereof. As a non-limiting example a software update may incorporate a new mitochondrial outcome that relates to a modified edible. Additionally or alternatively, the updated machine learning model may be transmitted to the remote device, wherein the remote device may replace the nourishment machine-learning model with the updated machine-learning model and develop the nourishment program as a function of the mitochondrial outcome using the updated machine-learning model. The updated machine-learning model may be transmitted by the remote device and received by computing device 104 as a software update, firmware update, or corrected nourishment machine-learning model. For example, and without limitation nourishment machine-learning model may utilize a neural net machine-learning process, wherein the updated machine-learning model may incorporate decision tree machine-learning processes.

Now referring to FIG. 2, an exemplary embodiment 200 of an epigenetic element 204 is illustrated. As used in this disclosure an “epigenetic element” is an element relating to the change in a gene activity and/or expression as a function of one or more external factors. For example, and without limitation, epigenetic element 204 may include the modification of one or more gene activities and/or expressions as a function of a modification of chromosomal regions. As a further non-limiting example, epigenetic element 204 may include the methylation of DNA as a function of an external factor that prevents certain genes from being expressed. As a further non-limiting example, epigenetic element 204 may include the alteration and/or modification of one or more gene activities and/or expressions as a function of a histone modification. Epigenetic element 204 may relate to a change in gene activity and/or expression as a function of an illicit drug 208. As used in this disclosure an “illicit drug” is a drug that is illegal to consume, sell, make, and/or otherwise interact with due to its addictive and/or harmful effects. For example, and without limitation, illicit drug 208 may include heroin, cocaine, methamphetamine, crack cocaine, LSD, ecstasy, PCP, angel dust, Krokodil, Molly, Flakka, fentanyl, ketamine, rohypnol, GHB, psilocybin, salvia, synthetic cannabinoids, synthetic cathinones, mescaline, DMT, and the like thereof. Epigenetic element 204 may relate to a change in gene activity and/or expression as a function of a medical background 212. As used in this disclosure a “medical background” is background of an individual's medical records. For example, and without limitation, medical background 212 may indicate that an individual has a dependency on opioids and/or pain medications. As a further non-limiting example, medical background 212 may indicate that an individual had a systemic bacterial and/or viral infection that left residual DNA damage. As a further non-limiting example, medical background 212 may indicate one or more traumatic injuries and/or experiences such as a car accident and/or mental abuse. Epigenetic element 204 may relate to a change in gene activity and/or expression as a function of an environmental influence 216. As used in this disclosure an “environmental influence” is an influence on an individual's DNA as a function of one or more environments and/or elements form an environment. For example, and without limitation, environmental influence 216 may include one or more DNA modifications as a function of oxalate presence in plants. As a further non-limiting example, environmental influence 216 may include one or more DNA modifications as a function of UV rays and/or harmful light exposures. As a further non-limiting example, environmental influence 216 may include one or more DNA modifications as a function of air and/or water pollution.

Now referring to FIG. 3, an exemplary embodiment 300 of an edible directory 304 according to an embodiment of the invention is illustrated. Edible directory 304 may be implemented, without limitation, as a relational databank, a key-value retrieval databank such as a NOSQL databank, or any other format or structure for use as a databank that a person skilled in the art would recognize as suitable upon review of the entirety of this disclosure. Edible directory 304 may alternatively or additionally be implemented using a distributed data storage protocol and/or data structure, such as a distributed hash table or the like. Edible directory 304 may include a plurality of data entries and/or records as described above. Data entries in a databank may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a databank may store, retrieve, organize, and/or reflect data and/or records as used herein, as well as categories and/or populations of data consistently with this disclosure. Edible directory 304 may include a carbohydrate tableset 308. Carbohydrate tableset 308 may relate to a nourishment composition of an edible with respect to the quantity and/or type of carbohydrates in the edible. As a non-limiting example, carbohydrate tableset 308 may include monosaccharides, disaccharides, oligosaccharides, polysaccharides, and the like thereof. Edible directory 304 may include a fat tableset 312. Fat tableset 312 may relate to a nourishment composition of an edible with respect to the quantity and/or type of esterified fatty acids in the edible. Fat tableset 312 may include, without limitation, triglycerides, monoglycerides, diglycerides, phospholipids, sterols, waxes, and free fatty acids. Edible directory 304 may include a fiber tableset 316. Fiber tableset 316 may relate to a nourishment composition of an edible with respect to the quantity and/or type of fiber in the edible. As a non-limiting example, fiber tableset 316 may include soluble fiber, such as beta-glucans, raw guar gum, psyllium, inulin, and the like thereof as well as insoluble fiber, such as wheat bran, cellulose, lignin, and the like thereof. Edible directory 304 may include a mineral tableset 320. Mineral tableset 320 may relate to a nourishment composition of an edible with respect to the quantity and/or type of minerals in the edible. As a non-limiting example, mineral tableset 320 may include calcium, phosphorous, magnesium, sodium, potassium, chloride, sulfur, iron, manganese, copper, iodine, zing, cobalt, fluoride, selenium, and the like thereof. Edible directory 304 may include a protein tableset 324. Protein tableset 324 may relate to a nourishment composition of an edible with respect to the quantity and/or type of proteins in the edible. As a non-limiting example, protein tableset 324 may include amino acids combinations, wherein amino acids may include, without limitation, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, and the like thereof. Edible directory 304 may include a vitamin tableset 328. Vitamin tableset 328 may relate to a nourishment composition of an edible with respect to the quantity and/or type of vitamins in the edible. As a non-limiting example, vitamin tableset 328 may include vitamin A, vitamin B₁, vitamin B₂, vitamin B₃, vitamin B₅, vitamin B₆, vitamin B₇, vitamin B₉, vitamin B₁₂, vitamin C, vitamin D, vitamin E, vitamin K, and the like thereof.

Now referring to FIG. 4, an exemplary embodiment 400 of a medical examination 120 is illustrated. Medical examination 120 may analyze and/or examine a cell 404. As used in this disclosure a “cell” is the smallest functional unit of life in an individual's body. For example, cell 404 may include one or more stem cells, red blood cells, white blood cells, platelets, nerve cells, muscle cells, cartilage cells, bone cells, skin cells, endothelial cells, epithelial cells, fat cells, sex cells, and the like thereof. Cell 404 may include a nucleus 408. As used in this disclosure a “nucleus” is a membrane-bound organelle in the cell that contains the cell's chromosomes. For example, and without limitation, nucleus 408 may include a nuclear envelope, nuclear pores, nuclear lamina, chromosomes, nucleolus, Cajal bodies, PIKA domains, PTF domains, splicing speckles, paraspeckles, perichromatin fibrils, clastosomes, and the like thereof. In an embodiment nucleus 408 may include a nuclear deoxyribonucleic acid 412. As used in this disclosure “nuclear deoxyribonucleic acid” is a nucleic acid, a polymeric biomolecule, and/or biopolymer found in the nucleus of cell 404. In an embodiment, and without limitation, nuclear deoxyribonucleic acid 412 may include a double helix structure with a first strand and a second strand wound around each other. Nuclear deoxyribonucleic acid 412 may include a nucleotide composed of a five-carbon sugar, a phosphate group, and/or an organic base, wherein the organic base may be comprised of purines, such as adenine and/or guanine, and/or pyrimidines, such as thymine and/or cytosine. Additionally or alternatively, cell 404 may include a mitochondrion 416. As used in this disclosure a “mitochondrion” is an organelle found in cell 404 that may perform biochemical processes associated with respiration and/or energy production. In an embodiment, mitochondria 416 may include an organelle associated with one or more functions associated with energy conversion, such as the conversion of pyruvate, glucose, and NADH to ATP, storage of calcium, cellular proliferation regulation, and the like thereof. In another embodiment, mitochondria 416 may include an outer membrane, an intermediate membrane, an inner mitochondrial membrane, a cristae space, and/or a matrix. Mitochondria 416 may include a mitochondrial deoxyribonucleic acid 420. As used in this disclosure a “mitochondrial deoxyribonucleic acid” is a nucleic acid, a polymeric biomolecule, and/or biopolymer found in the mitochondria of cell 404. In an embodiment mitochondrial deoxyribonucleic acid 420 may be comprised of a closed and/or circular structure. In yet another embodiment, mitochondrial deoxyribonucleic acid 420 may be comprised of 16,569 nucleotides. Mitochondrial deoxyribonucleic acid 420 may be a haploid structure that is inherited only from the mother of the individual.

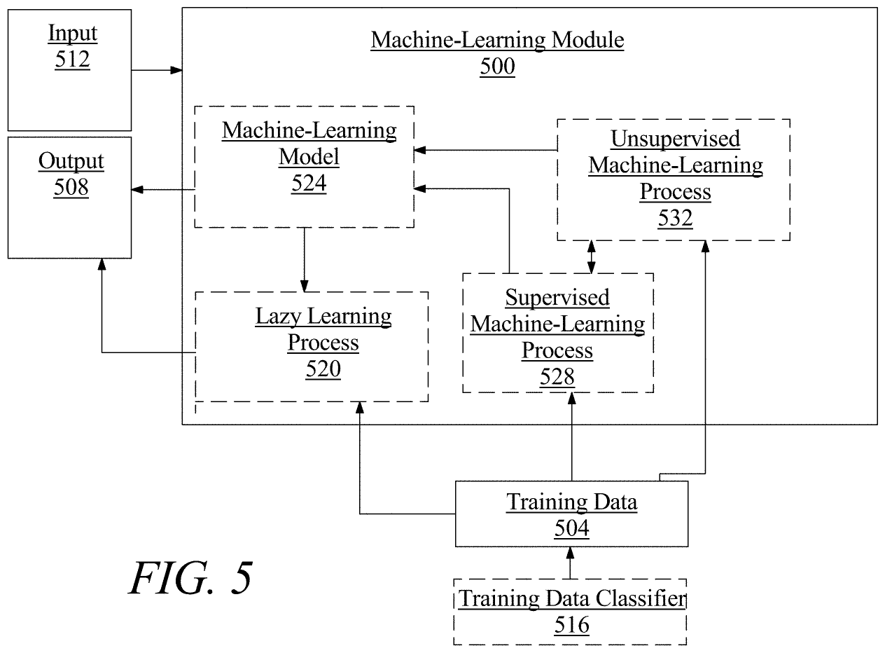

Referring now to FIG. 5, an exemplary embodiment of a machine-learning module 500 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 504 to generate an algorithm that will be performed by a computing device/module to produce outputs 508 given data provided as inputs 512; this is in contrast to a non-machine learning software program where the commands to be executed are determined in advance by a user and written in a programming language.

Still referring to FIG. 5, “training data,” as used herein, is data containing correlations that a machine-learning process may use to model relationships between two or more categories of data elements. For instance, and without limitation, training data 504 may include a plurality of data entries, each entry representing a set of data elements that were recorded, received, and/or generated together; data elements may be correlated by shared existence in a given data entry, by proximity in a given data entry, or the like. Multiple data entries in training data 504 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 504 according to various correlations; correlations may indicate causative and/or predictive links between categories of data elements, which may be modeled as relationships such as mathematical relationships by machine-learning processes as described in further detail below. Training data 504 may be formatted and/or organized by categories of data elements, for instance by associating data elements with one or more descriptors corresponding to categories of data elements. As a non-limiting example, training data 504 may include data entered in standardized forms by persons or processes, such that entry of a given data element in a given field in a form may be mapped to one or more descriptors of categories. Elements in training data 504 may be linked to descriptors of categories by tags, tokens, or other data elements; for instance, and without limitation, training data 504 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 5, training data 504 may include one or more elements that are not categorized; that is, training data 504 may not be formatted or contain descriptors for some elements of data. Machine-learning algorithms and/or other processes may sort training data 504 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 504 to be made applicable for two or more distinct machine-learning algorithms as described in further detail below. Training data 504 used by machine-learning module 500 may correlate any input data as described in this disclosure to any output data as described in this disclosure. As a non-limiting illustrative example inputs of biological indicators and probabilistic vectors may be inputs, wherein an output may be a mitochondrial profile.

Further referring to FIG. 5, training data may be filtered, sorted, and/or selected using one or more supervised and/or unsupervised machine-learning processes and/or models as described in further detail below; such models may include without limitation a training data classifier 516. Training data classifier 516 may include a “classifier,” which as used in this disclosure is a machine-learning model as defined below, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Machine-learning module 500 may generate a classifier using a classification algorithm, defined as a processes whereby a computing device and/or any module and/or component operating thereon derives a classifier from training data 504. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 516 may classify elements of training data to sub-categories of probabilistic vectors such as a probability as a function of an inheritance element and/or or a probability as a function of a mutation component.

Still referring to FIG. 5, machine-learning module 500 may be configured to perform a lazy-learning process 520 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 504. Heuristic may include selecting some number of highest-ranking associations and/or training data 504 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 5, machine-learning processes as described in this disclosure may be used to generate machine-learning models 524. A “machine-learning model,” as used in this disclosure, is a mathematical and/or algorithmic representation of a relationship between inputs and outputs, as generated using any machine-learning process including without limitation any process as described above, and stored in memory; an input is submitted to a machine-learning model 524 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 524 may be generated by creating an artificial neural network, such as a convolutional neural network comprising an input layer of nodes, one or more intermediate layers, and an output layer of nodes. Connections between nodes may be created via the process of “training” the network, in which elements from a training data 504 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 5, machine-learning algorithms may include at least a supervised machine-learning process 528. At least a supervised machine-learning process 528, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may include biological indicators and/or probabilistic vectors as described above as inputs, mitochondrial profiles as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 504. Persons skilled in the art, upon reviewing the entirety of this disclosure, will be aware of various possible variations of at least a supervised machine-learning process 528 that may be used to determine relation between inputs and outputs. Supervised machine-learning processes may include classification algorithms as defined above.

Further referring to FIG. 5, machine learning processes may include at least an unsupervised machine-learning processes 532. An unsupervised machine-learning process, as used herein, is a process that derives inferences in datasets without regard to labels; as a result, an unsupervised machine-learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

Still referring to FIG. 5, machine-learning module 500 may be designed and configured to create a machine-learning model 524 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 5, machine-learning algorithms may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

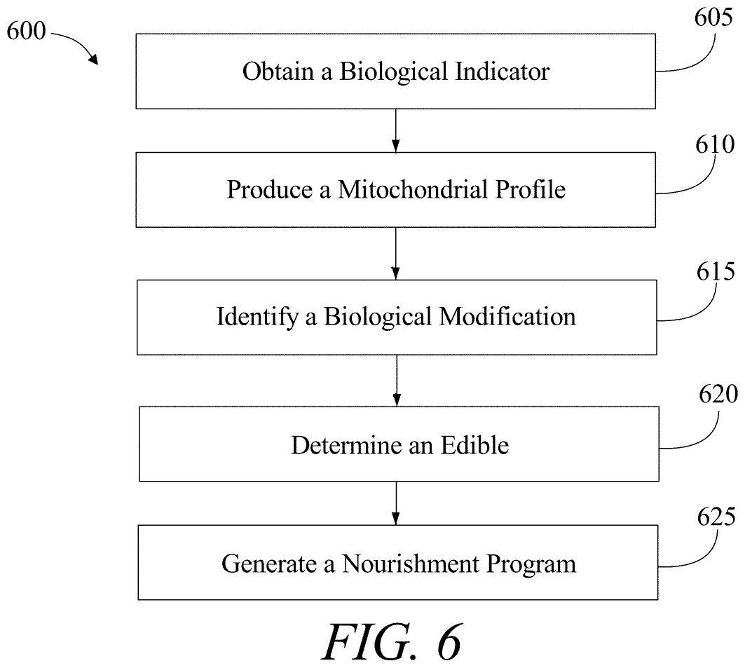

Now referring to FIG. 6, an exemplary embodiment of a method 600 for generating a mitochondrial dysfunction nourishment program is illustrated. At step 605, a computing device 104 obtains a biological indicator 108. Computing device 104 includes any of the computing device 104 as described above, in reference to FIGS. 1-5. Biological indicator 108 includes any of the biological indicator 108 as described above, in reference to FIGS. 1-5.

Still referring to FIG. 6, at step 610, computing device 104 produces a mitochondrial profile 112 as a function of biological indicator 108. Mitochondrial profile 112 includes any of the mitochondrial profile 112 as described above, in reference to FIGS. 1-5. Computing device 104 produces mitochondrial profile 112 by identifying a probabilistic vector 116 as a function of a medical examination 120. Probabilistic vector 116 includes any of the probabilistic vector 116 as described above, in reference to FIGS. 1-5. Medical examination 120 includes any of the medical examination 120 as described above, in reference to FIGS. 1-5. Computing device 104 produces mitochondrial profile 112 as a function of probabilistic vector 116 and biological indicator 108 using a profile machine-learning model 124. Profile machine-learning model 124 includes any of the profile machine-learning model 124 as described above, in reference to FIGS. 1-5.

Still referring to FIG. 6, at step 615, computing device 104 identifies a biological modification 128 as a function of mitochondrial profile 112. Biological modification 128 includes any of the biological modification 128 as described above, in reference to FIGS. 1-5. Computing device 104 identifies biological modification 128 by receiving a medical guideline 132. Medical guideline 132 includes any of the medical guideline 132 as described above, in reference to FIGS. 1-5. Computing device identifies biological modification 128 as a function of medical guideline 132 and mitochondrial profile 112 using a biological machine-learning model 136. Biological machine-learning model 136 includes any of the biological machine-learning model 136 as described above, in reference to FIGS. 1-5.

Still referring to FIG. 6, at step 620, computing device 104 determines an edible 140 as a function of biological modification 128. Edible 140 includes any of the edible 140 as described above, in reference to FIGS. 1-5.

Still referring to FIG. 6, at step 625, computing device 104 generates a nourishment program 144 as a function of edible 140. Nourishment program 144 includes any of the nourishment program 144 as described above, in reference to FIGS. 1-5.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

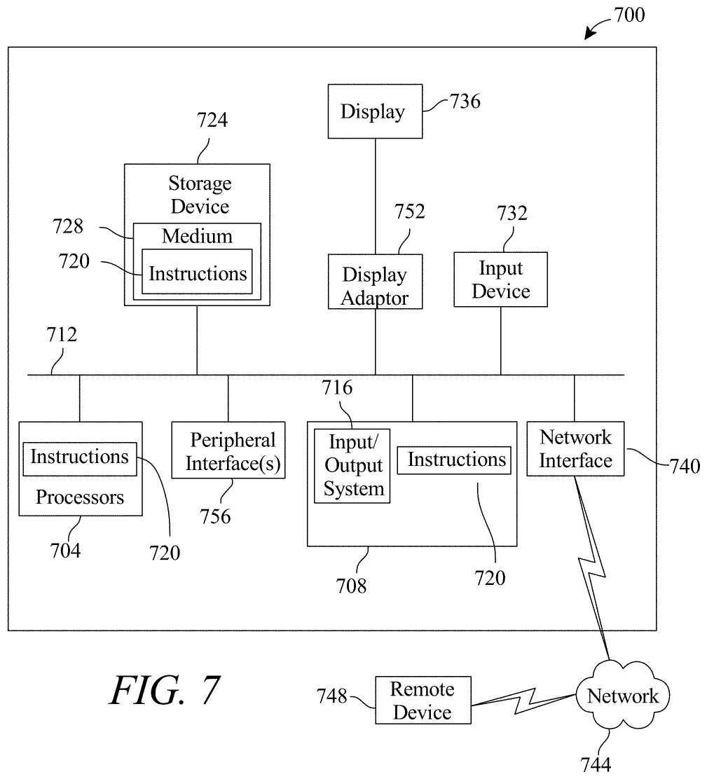

FIG. 7 shows a diagrammatic representation of one embodiment of a computing device in the exemplary form of a computer system 700 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 700 includes a processor 704 and a memory 708 that communicate with each other, and with other components, via a bus 712. Bus 712 may include any of several types of bus structures including, but not limited to, a memory bus, a memory controller, a peripheral bus, a local bus, and any combinations thereof, using any of a variety of bus architectures.

Processor 704 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 704 may be organized according to Von Neumann and/or Harvard architecture as a non-limiting example. Processor 704 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC)

Memory 708 may include various components (e.g., machine-readable media) including, but not limited to, a random-access memory component, a read only component, and any combinations thereof. In one example, a basic input/output system 716 (BIOS), including basic routines that help to transfer information between elements within computer system 700, such as during start-up, may be stored in memory 708. Memory 708 may also include (e.g., stored on one or more machine-readable media) instructions (e.g., software) 720 embodying any one or more of the aspects and/or methodologies of the present disclosure. In another example, memory 708 may further include any number of program modules including, but not limited to, an operating system, one or more application programs, other program modules, program data, and any combinations thereof.

Computer system 700 may also include a storage device 724. Examples of a storage device (e.g., storage device 724) include, but are not limited to, a hard disk drive, a magnetic disk drive, an optical disc drive in combination with an optical medium, a solid-state memory device, and any combinations thereof. Storage device 724 may be connected to bus 712 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 724 (or one or more components thereof) may be removably interfaced with computer system 700 (e.g., via an external port connector (not shown)). Particularly, storage device 724 and an associated machine-readable medium 728 may provide nonvolatile and/or volatile storage of machine-readable instructions, data structures, program modules, and/or other data for computer system 700. In one example, software 720 may reside, completely or partially, within machine-readable medium 728. In another example, software 720 may reside, completely or partially, within processor 704.

Computer system 700 may also include an input device 732. In one example, a user of computer system 700 may enter commands and/or other information into computer system 700 via input device 732. Examples of an input device 732 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 732 may be interfaced to bus 712 via any of a variety of interfaces (not shown) including, but not limited to, a serial interface, a parallel interface, a game port, a USB interface, a FIREWIRE interface, a direct interface to bus 712, and any combinations thereof. Input device 732 may include a touch screen interface that may be a part of or separate from display 736, discussed further below. Input device 732 may be utilized as a user selection device for selecting one or more graphical representations in a graphical interface as described above.

A user may also input commands and/or other information to computer system 700 via storage device 724 (e.g., a removable disk drive, a flash drive, etc.) and/or network interface device 740. A network interface device, such as network interface device 740, may be utilized for connecting computer system 700 to one or more of a variety of networks, such as network 744, and one or more remote devices 748 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 744, may employ a wired and/or a wireless mode of communication. In general, any network topology may be used. Information (e.g., data, software 720, etc.) may be communicated to and/or from computer system 700 via network interface device 740.

Computer system 700 may further include a video display adapter 752 for communicating a displayable image to a display device, such as display device 736. Examples of a display device include, but are not limited to, a liquid crystal display (LCD), a cathode ray tube (CRT), a plasma display, a light emitting diode (LED) display, and any combinations thereof. Display adapter 752 and display device 736 may be utilized in combination with processor 704 to provide graphical representations of aspects of the present disclosure. In addition to a display device, computer system 700 may include one or more other peripheral output devices including, but not limited to, an audio speaker, a printer, and any combinations thereof. Such peripheral output devices may be connected to bus 712 via a peripheral interface 756. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve systems and methods according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A system for generating a mitochondrial dysfunction nourishment program, the system comprising:
a computing device, the computing device configured to:
obtain a biological indicator;
produce a mitochondrial profile as a function of the biological indicator, wherein producing the mitochondrial profile further comprises:
identifying a probabilistic vector as a function of a medical examination; and
producing the mitochondrial profile as a function of the probabilistic vector and the biological indicator using a profile machine-learning model;
identify a biological modification as a function of the mitochondrial profile, wherein identifying the biological modification further comprises;
receiving a medical guideline; and
identifying the biological modification as a function of the medical guideline and mitochondrial profile using a biological machine-learning model;
determine an edible as a function of the biological modification; and
generate a nourishment program as a function of the edible.

2. The system of claim 1, wherein the biological indicator includes an inheritance element.

3. The system of claim 1, wherein obtaining the biological indicator further comprises identifying a mutation component and obtaining the biological indicator as a function of the mutation component.

4. The system of claim 3, wherein identifying the mutation component further comprises:
identifying a spontaneity element;
determining a mutation rate as a function of the spontaneity element and a mutation grouping; and
identifying the mutation component as a function of the mutation rate.

5. The system of claim 3, wherein the mutation component includes an epigenetic element.

6. The system of claim 1, wherein producing the mitochondrial profile further comprises determining a mitochondrial dysfunction and producing the mitochondrial profile as a function of the mitochondrial dysfunction.

7. The system of claim 1, wherein producing the mitochondrial profile further comprises:
identifying a first probabilistic vector as a function of a first medical examination;
receiving a second medical examination as a function of a follow-up recommendation;
generating a second probabilistic vector as a function of the second medical examination; and
producing the mitochondrial profile as a function of the first probabilistic vector and the second probabilistic vector using the profile machine-learning model.

8. The system of claim 1, wherein identifying the probabilistic vector further comprises:
obtaining a mitochondrial deoxyribonucleic acid vector;
receiving a nuclear deoxyribonucleic acid vector; and
identifying the probabilistic vector as a function of the mitochondrial deoxyribonucleic acid vector and the nuclear deoxyribonucleic acid vector.

9. The system of claim 1, wherein determining the edible further comprises:
receiving a nourishment composition from an edible directory;
producing a nourishment demand as a function of the biological modification; and
determining the edible as a function of the nourishment composition and the nourishment demand using an edible machine-learning model.

10. The system of claim 1, wherein generating the nourishment program further comprises:
receiving a mitochondrial outcome; and
generating the nourishment program as a function of the mitochondrial outcome using a nourishment machine-learning model.

11. A method for generating a mitochondrial dysfunction nourishment program, the method comprising:
obtaining, by a computing device, a biological indicator;
producing, by the computing device, a mitochondrial profile as a function of the biological indicator, wherein producing the mitochondrial profile further comprises:
identifying a probabilistic vector as a function of a medical examination; and
producing the mitochondrial profile as a function of the probabilistic vector and the biological indicator using a profile machine-learning model;
identifying, by the computing device, a biological modification as a function of the mitochondrial profile, wherein identifying the biological modification further comprises;
receiving a medical guideline; and
identifying the biological modification as a function of the medical guideline and mitochondrial profile using a biological machine-learning model;
determining, by the computing device, an edible as a function of the biological modification;
and
generating, by the computing device, a nourishment program as a function of the edible.

12. The method of claim 11, wherein the biological indicator includes an inheritance element.

13. The method of claim 11, wherein obtaining the biological indicator further comprises identifying a mutation component and obtaining the biological indicator as a function of the mutation component.

14. The method of claim 13, wherein identifying the mutation component further comprises:
identifying a spontaneity element;
determining a mutation rate as a function of the spontaneity element and a mutation grouping; and
identifying the mutation component as a function of the mutation rate.

15. The method of claim 13, wherein the mutation component includes an epigenetic element.

16. The method of claim 11, wherein producing the mitochondrial profile further comprises determining a mitochondrial dysfunction and producing the mitochondrial profile as a function of the mitochondrial dysfunction.

17. The method of claim 11, wherein producing the mitochondrial profile further comprises:
identifying a first probabilistic vector as a function of a first medical examination;
receiving a second medical examination as a function of a follow-up recommendation;
generating a second probabilistic vector as a function of the second medical examination; and
producing the mitochondrial profile as a function of the first probabilistic vector and the second probabilistic vector using the profile machine-learning model.

18. The method of claim 11, wherein identifying the probabilistic vector further comprises:
obtaining a mitochondrial deoxyribonucleic acid vector;
receiving a nuclear deoxyribonucleic acid vector; and
identifying the probabilistic vector as a function of the mitochondrial deoxyribonucleic acid vector and the nuclear deoxyribonucleic acid vector.

19. The method of claim 11, wherein determining the edible further comprises:
receiving a nourishment composition from an edible directory;
producing a nourishment demand as a function of the biological modification; and
determining the edible as a function of the nourishment composition and the nourishment demand using an edible machine-learning model.

20. The method of claim 11, wherein generating the nourishment program further comprises:
receiving a mitochondrial outcome; and
generating the nourishment program as a function of the mitochondrial outcome using a nourishment machine-learning model.
